(19) United States
(12) Patent Application Publication
(10) Pub. No.: US 2017/0100279 A1
(43) Pub. Date: Apr. 13, 2017

(54) METHOD AND APPARATUS FOR SECURING AN OBJECT TO A HUMAN BODY FOR THERAPEUTIC PURPOSES

(71) Applicant: Jeff Ott, Sylvania, OH (US)

(72) Inventor: Jeff Ott, Sylvania, OH (US)

(21) Appl. No.: 15/290,426

(22) Filed: Oct. 11, 2016

Related U.S. Application Data
(60) Provisional application No. 62/239,516, filed on Oct. 9, 2015.

(51) Int. Cl. A61F 7/02; A61F 7/10

(52) U.S. Cl. CPC A61F 7/02; A61F 7/10; A61F 2007/023; A61F 2007/0249; A61F 2007/108

(57) ABSTRACT

An apparatus and a method of use for securing an object to a human body comprising: a first belt, wherein the first belt further comprises a central portion; a second belt, wherein the second belt further comprises a pouch for holding an associated ice pack; and, an associated ice pack; wherein the central portion of the first belt comprises insulating material with a first bolster and a second bolster; and, wherein the first and second belt further comprise a fastening means to secure the first belt and second belt to a human body.

### TECHNICAL FIELD

The present invention relates to an apparatus for securing an item next to a human body. More particularly, the present invention is directed at a therapeutic device for applying and securing an ice pack in order to provide ice therapy to a portion of the human body.

### BACKGROUND

Conventional approaches to treating back pain have long included the use of prescription medications, surgery, hot and cold therapy, chiropractic manipulation, physical therapy, or a combination of some or all of these methods. Surgery can be successful at reducing or eliminating back pain, but surgery is invasive, expensive, and can lead to complications and other adverse side effects.

Medications are commonly prescribed to manage the pain symptoms, but in most cases they do not treat any underlying cause of the pain. Medications can also cause allergic reactions and other adverse side effects, and in the case of some painkillers, the medications themselves can be habit forming and addictive. Furthermore, persons who have suffered from dependence in the past cannot use many of the most commonly prescribed medications.

Current therapeutic solutions consist of applying heat or cold over a broad area, taking the generic approach that heating or cooling the entire area offers some benefit to the affected area. Furthermore, current broadly applied cold therapy products often don't get cold enough or retain their cold temperatures long enough to provide meaningful reduction in inflammation.

What is needed is a non-invasive, non-surgical, medication free, therapeutic solution that can apply cold therapy that targets pain sources such as inflamed, displaced, or herniated discs, and/or swollen muscles and tissue in and around the spinal column. Additionally, this solution needs to reach lower temperatures and retain these temperatures long enough to supply meaningful reduction in inflammation, thereby treating the cause of and reducing back pain.

### SUMMARY

Provided is an apparatus and a method of use for securing an object to a human body comprising: a first belt, wherein the first belt further comprises a central portion; a second belt, wherein the second belt further comprises a pouch for holding an associated ice pack; and, an associated ice pack; wherein the central portion of the first belt comprises insulating material with a first bolster and a second bolster; and, wherein the first and second belt further comprise a fastening means to secure the first belt and second belt to a human body.

### DESCRIPTION OF THE DRAWINGS

FIG. 1 shows an embodiment of the present invention from the outer side of the securing apparatus with two interchangeable ice packs;

FIG. 2 shows an embodiment of the securing apparatus from above;

FIG. 3 shows a close up of an embodiment of a first bolster;

FIG. 4 shows a close up of an embodiment of a second bolster;

FIG. 5 shows an embodiment of the present invention from the inner side of the securing apparatus;

FIG. 6 shows a close up of an embodiment of the pouch with the ice pack partially inserted therein;

FIG. 7 shows a close up of the pouch and ice pack with the ice pack removed.

### DETAILED DESCRIPTION

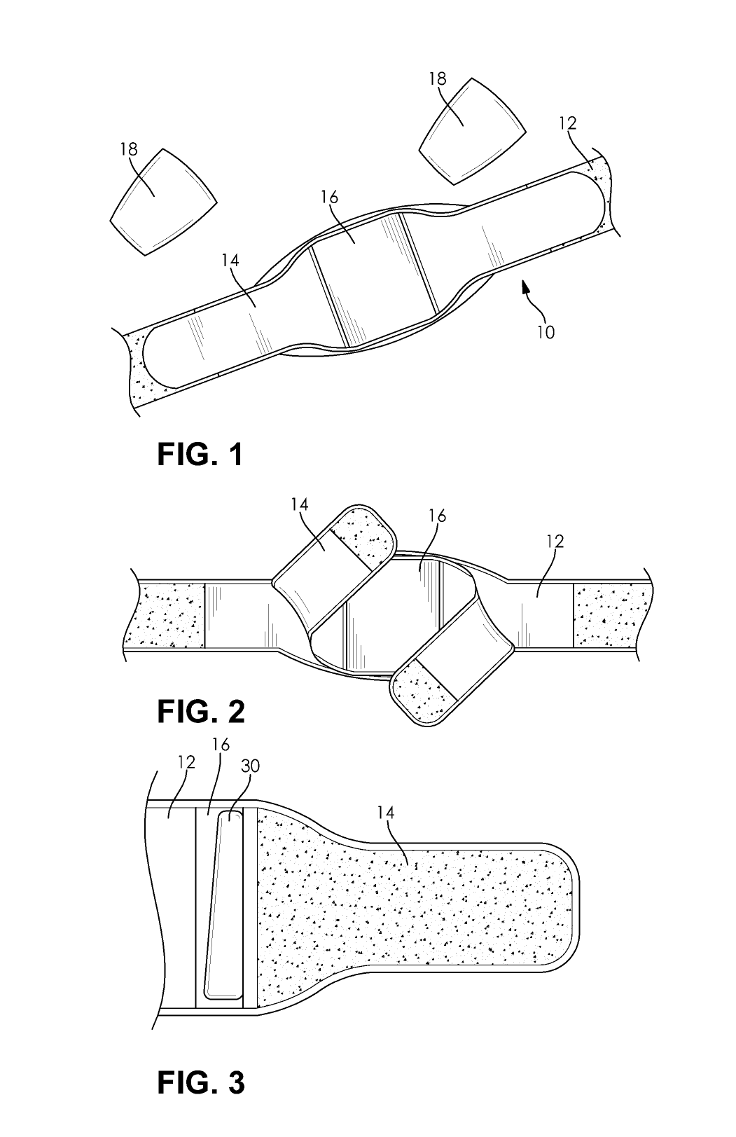

With reference to FIGS. 1 and 2, an embodiment of the securing apparatus can consist of a two-piece belt 10 which can be fixedly connected in the center. The inner belt 12 is closest to the user while the apparatus is being worn, while the outer belt 14 is positioned exterior to the inner belt 12 while the apparatus is being worn. The two-piece belt 10 can be secured to the user by fastening means, which can include hook and loop closures at the distal ends of the inner belt 12 and outer belt 14. The inner belt 12 can have loop closures on a first side with hook closures on a second side. The outer belt 14 can have loop closures on a first side corresponding to the hook closures on the second side of the inner belt 12.

With continued reference to FIGS. 1 and 2, the outer belt 14 can be made of a flexible material. According to one embodiment, the outer belt 14 can be made of neoprene based foam with a knit textile covering commonly known as 2-sided wetsuit neoprene. The edges of the inner and outer belts 12, 14, can be bound with a stretchable knit binding. According to one embodiment, the binding can be polyester or nylon based with spandex fibers. The outer belt 14 can be fixed to the inner belt 12 by stitching the two together in the middle. The center portion 16 of the outer belt 14 can be made from a laminated polyethylene based foam that can be covered with a knit textile on one or both sides. The inner belt 12 can be made of flexible mesh material that can be polyester or nylon based.

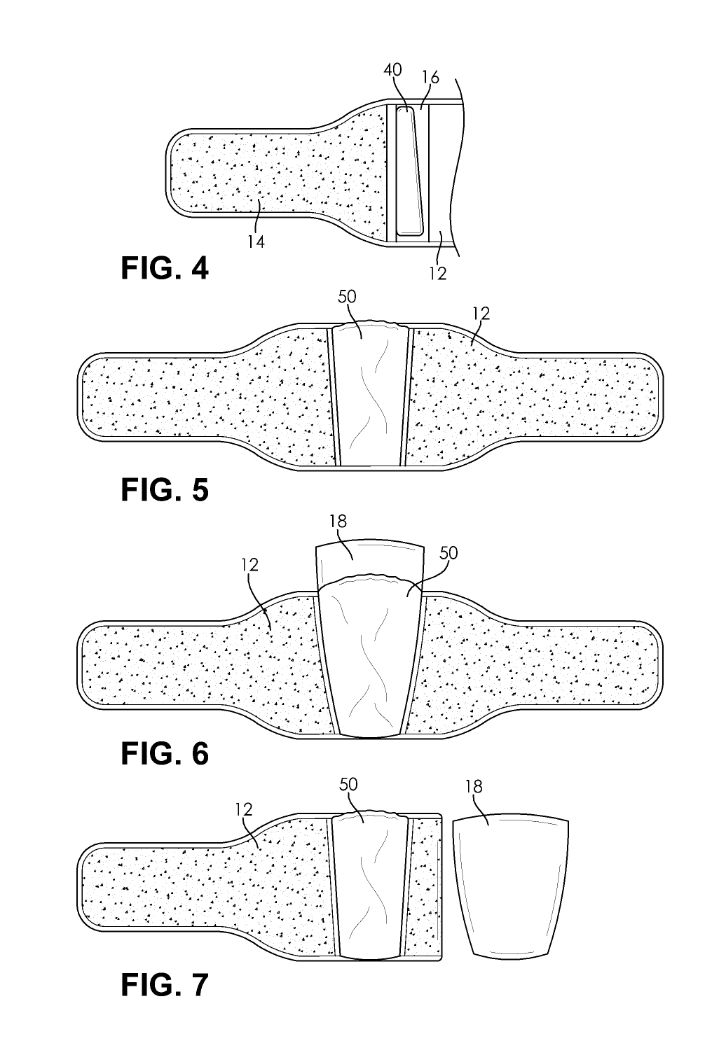

With reference to FIGS. 3 and 4, the inner belt 12 is folded away to reveal the inside of the outer belt 14 and the center portion 16. The inside face of the center portion 16 can include a first bolster 30 and a second bolster 40 that can be molded as a part of the center portion 16. The unique shape of the bolsters 30, 40 corresponds to the shape of the ice pack 18 (shown in FIG. 1). Specifically, the lower portion of the first and second bolsters 30, 40 can be wider than the upper portion, thereby helping the ice pack 18 retain its shape while in use. The construction of the center portion 16 and the bolsters 30, 40 can serve to insulate the ice pack 18 from the environment which can allow the ice pack 18 to retain its cold temperature for a longer period of time, thereby increasing the therapeutic application time.

With reference to FIGS. 5-7, the inner belt 12 can have a pouch 50 that can be fixedly attached to the inner belt 12 on three sides, leaving a fourth side open to receive the associated ice pack 18 (shown partially inserted into the pouch 50 in FIG. 6). The pouch 50 can have a generally trapezoidal shape which can correspond to the shape of the ice pack 18 to assure a snug fit and to further assist in maintaining the shape of the ice pack 18 while in use. The generally trapezoidal shape of the ice pack 18 and the associated pouch 50 can allow the effect of the cold therapy to be directed specifically to the spinal column of the user, thereby increasing effectiveness of the placement of the ice pack 18 against the user's body. The pouch 50 can be made of a knit textile similar to that used for covering the center portion 16 of the outer belt 14. According to one embodiment of the invention, the pouch 50 can be made of a stretchable fleece material, such as polyester brushed knit containing lycra or spandex.

With reference to FIGS. 1 and 7, the ice pack 18 can be a sealed plastic container filled with a glycol/water mixture that changes phase at approximately 5° Fahrenheit. This mixture can allow the ice pack 18 to be chilled in a conventional freezer at temperatures below 32° Fahrenheit while maintaining the pliability of the ice pack 18. The mass of the ice pack 18 can be such that the ice pack maintains its lower temperature for a longer period of time. According to one embodiment, the mass of the ice pack 18 can be approximately 25 ounces. The shape of the ice pack can be generally trapezoidal to correspond with the unique shape of the pouch 50 and bolsters 30, 40.

In order to properly utilize the two-piece belt 10 for cold therapy relief, the ice pack 18 can be chilled in a conventional freezer at temperatures below 32° Fahrenheit for a period between 4 to 8 hours. At this point, the user can remove the ice pack 18 from the freezer and insert the ice pack 18 into the pouch 50 with such an orientation as to match the shape of the ice pack 18 to the shape of the pouch 50. Once the ice pack 18 is fully inserted into the pouch 50, the user places the two-piece belt 10 around his or her waist, making certain to position the pouch 50 containing the ice pack 18 over the desired treatment area, and fastens the inner belt 12 using the hook and loop fasteners. Once the inner belt 12 is properly placed and fastened, the outer belt 14 can be adjusted for comfort and fastened to the inner belt 12 using the hook and loop closures. In this position, the two-piece belt 10 delivers concentrated cold therapy directly to the spinal column to treat the source of pain. The insulating function of the foam that comprises the center portion 16 of the two-piece belt 10 and the larger mass of the ice pack 18 can prevent cold loss to the environment. According to one embodiment, the ice pack 18 can retain therapeutically effective temperatures for up to 40 minutes, which can be up to double the maximum amount of time (20 minutes) recommended for meaningful reduction in inflammation and pain relief

The bolsters 30, 40, and the shape of the pouch 50 can function to maintain the shape of the ice pack 18 to prevent the ice pack 18 from flattening out which keeps the cold therapy treatment focused on the desired treatment area and minimizes cold loss to surrounding tissues and structures that are not intended to be treated. In a preferred version of the invention, the distance between the bolsters 30, 40, is less than the width of the ice pack 18 when it is flattened out.

The inner belt 12 and outer belt 14 of the two-piece belt 10 also provide the added benefit of providing support to the back and spinal column during therapeutic treatment.

When used according to the claimed method, the present invention can provide a longer lasting, more meaningful reduction in inflammation and pain without the use of medications, the intrusiveness of surgery, or the side effects associated with these methods of treatment.

Having thus described the invention, it is now claimed:

I claim:

1. An apparatus for securing an object to a human body comprising:
a. a first belt, wherein the first belt further comprises a central portion;
b. a second belt, wherein the second belt further comprises a pouch for holding an associated ice pack and,
c. an associated ice pack;
wherein the central portion of the first belt comprises insulating material with a first bolster and a second bolster; and, wherein the first and second belt further comprise a fastening means to secure the first belt and second belt to a human body.

2. The apparatus of claim 1 wherein the central portion further comprises a laminated polyethylene based insulating foam.

3. The apparatus of claim 2 wherein the polyethylene based insulating foam is covered on at least one side with a knit material.

4. The apparatus of claim 3 wherein the ice pack is generally trapezoidal in shape.

5. The apparatus of claim 4 wherein the pouch, first bolster, and second bolster are appropriately shaped corresponding to the general trapezoidal shape of the ice pack.

6. The apparatus of claim 1 wherein the ice pack is generally trapezoidal in shape.

7. The apparatus of claim 6 wherein the pouch, first bolster, and second bolster are appropriately shaped corresponding to the general trapezoidal shape of the ice pack.

8. The apparatus of claim 1 wherein the ice pack has a mass of at least 25 ounces.

9. The apparatus of claim 5 wherein the ice pack has a mass of at least 25 ounces.

10. The apparatus of claim 7 wherein the ice pack has a mass of at least 25 ounces.

11. A method of cold therapy comprising the steps of:
a. providing an apparatus for securing an object to a human body comprising:
i. a first belt, wherein the first belt further comprises a central portion;
ii. a second belt, wherein the second belt further comprises a pouch for holding an associated ice pack and,
iii. an associated ice pack;
wherein the central portion of the first belt comprises insulating material with a first bolster and a second bolster; and, wherein the first and second belt further comprise a fastening means to secure the first belt and second belt to a human body;
b. storing the ice pack at a temperature below 32° Fahrenheit for a period of at least 4 hours;
c. placing the chilled ice pack in the pouch;
d. securing the first belt to a user's body, wherein the ice pack is placed directly over a desired treatment area;
e. fastening the first belt and second belt around the user's body;
f wearing the apparatus for a period of at least 20 minutes;
g. removing the apparatus from the user's body and replacing the ice pack in storage below 32° Fahrenheit.

12. The method of claim 11 wherein the central portion of the provided apparatus further comprises a laminated polyethylene based insulating foam.

13. The method of claim 12 wherein the polyethylene based insulating foam is covered on at least one side with a knit material.

14. The method of claim 13 wherein the ice pack is generally trapezoidal in shape.

15. The method of claim 14 wherein the pouch, first bolster, and second bolster are appropriately shaped corresponding to the general trapezoidal shape of the ice pack.

16. The method of claim 11 wherein the ice pack is generally trapezoidal in shape.

17. The method of claim 16 wherein the pouch, first bolster, and second bolster are appropriately shaped corresponding to the general trapezoidal shape of the ice pack.

18. The method of claim 11 wherein the ice pack has a mass of at least 25 ounces.

19. The method of claim 15 wherein the ice pack has a mass of at least 25 ounces.

20. The method of claim 17 wherein the ice pack has a mass of at least 25 ounces.
